(19) United States
(12) Patent Application Publication
(10) Pub. No.: US 2006/0052297 A1
(43) Pub. Date: Mar. 9, 2006

(54) Method of up-regulating tumor antigen expression using thymalfasin

(76) Inventors: Guido Rasi, Rome (IT); Enrico Garaci, Rome (IT); Paolo Sinibaldi-Vallebona, Rome (IT)

Correspondence Address: ROTHWELL, FIGG, ERNST & MANBECK, P.C., 1425 K STREET, N.W., SUITE 800, WASHINGTON, DC, 20005, US

(21) Appl. No.: 10/519,082

(22) PCT Filed: Jun. 30, 2003
(86) PCT No.: PCT/US03/20829

§ 371 Date: Oct. 21, 2005

Related U.S. Application Data
(60) Provisional application No. 60/391,969, filed on Jun. 28, 2002.

(51) Int. Cl. A61K 38/17; A61K 31/47

(52) U.S. Cl. 514/12; 514/310

(57) ABSTRACT

The present invention provides a method for up-regulating tumor cell antigen expression, comprising administering to the cells an amount of thymalfasin sufficient to increase the expression of TLP relative to that of untreated tumor cells. Also provided are methods for enhancing the sensitivity of a immunodiagnostic or immunotherapeutic method, comprising pre-treating target tumor cells by administering to the cells an amount of thymalfasin sufficient to increase the expression of TLP relative to that of untreated tumor cells, followed by application of the immunodiagnostic or immunotherapeutic method. These methods are applicable to both in vivo and in vitro diagnostic methods, and to in vivo immunotherapeutic methods.

### RELATED APPLICATION DATA

This application claims the benefit of provisional application 60/391,969, filed Jun. 28, 2002.

### TECHNICAL FIELD

The invention relates to the field of cancer diagnostics and therapeutics, and to the use of thymalfasin (thymosin α-1) in the diagnosis and treatment of cancer.

### BACKGROUND

The development of anticancer vaccines, immuno-imaging and drug delivery techniques all depend on the identification of specific molecular targets (e.g., tumor antigens) that are available at the tumor cell membrane and are properly presented by MHC-1 molecules. Antigen identification has remained a central problem in tumor immunotherapy, and in cancer vaccine development in particular. While a variety of tumor-specific antigens have been identified, the level of expression of these antigens and/or their inconstant presentation by the MHC-1 molecules has been such that it has been difficult to develop immunotherapies, and immune-based diagnostic methods, of sufficient specificity and sensitivity to be able to detect the presence of, and destroy, cancer cells. Enhanced sensitivity for detection of tumor antigens permits diagnosis in early stages of the disease and treatment in early stages with fewer tumor cells, as well as more aggressive treatment of metastatic conditions.

Previously we have shown that TLP, a tumor specific antigen, is expressed in both human and experimental tumors, and has a conserved sequence. This makes TLP a good candidate for use as a target antigen for immuno diagnostic and immunotherapy methods, however, the level of expression of TLP, as with most of the tumor molecular targets, is less than ideal for these purposes.

Thymosin α-1, or thymalfasin is a 28-amino acid peptide that is normally found in the circulation. Thymalfasin stimulates thymocyte growth and differentiation, production of IL-2, T cell IL-2 receptors, IFNα and IFNγ. Baxevanis, 1990; Bepler, 1994; Serrate, 1987; Stzein, 1989. Thymosin alpha 1 also has been shown to up-regulate MHC Class I expression. Giuliani, 2000. Thymalfasin has been evaluated for its therapeutic potential in multiple diseases, including chronic hepatitis B and C, acquired immune deficiency syndrome (AIDS), depressed response to vaccination, and cancer. Andreone, 1996; Garaci, 1998, 2000; Gravenstein, 1986; Mutchnick, 1999; Sherman, 1998.

### SUMMARY OF THE INVENTION

It has been found that thymalfasin is able to up-regulate the expression of TLP on colorectal cancer cells, either in vitro or in vivo. This enhanced TLP expression makes possible a variety of improved diagnostic and therapeutic methods based on improved cancer cell targeting of, for example, radio-immuno guided surgery or immuno-scintigraphic techniques. The ability to enhance the expression of a tumor antigen with a non-toxic treatment such as thymalfasin could thus be quite useful for both therapeutic and diagnostic purposes, allowing earlier detection and treatment of cancers, as well as more aggressive and thorough treatment of advanced cancers, particularly metastatic cancers.

### BRIEF DESCRIPTION OF THE DRAWINGS

FIG. 1: TLP is expressed on the cell membrane of colorectal cancer cell line DHD-K12.

FIG. 2: TLP expression on the cell surface of colorectal cancer cell line WiDr is increased by treatment with thymalfasin.

FIG. 3: TLP expression on tumor cells from ascites is increased by treatment of animals with thymalfasin.

### DETAILED DESCRIPTION

The expression of TLP antigen can be up-regulated by treatment with thymalfasin. The increased expression could lead to more effective CTL responses from induction of a specific CD8 population. Additionally, this increased expression could allow cells to be better targeted and detected for radio-immuno guided surgery or immuno-scintigraphic techniques. Moreover, the availability of an experimental tumor naturally expressing a human antigen could be of great use in development of preclinical models. Enhancing tumor antigen expression with the present methods adds increased sensitivity to both diagnostic and immunotherapeutic methods, which in turn permits earlier detection and treatment of cancers, and a more aggressive and thorough treatment of advanced cancers, including metastatic cancers.

TLP expression-enhancing amounts of thymalfasin peptide can be determined by routine dose-titration experiments. Up-regulation of TLP can be achieved in vivo by administration of between 2 μg/kg and 6 mg/kg body weight of thymalfasin, preferably between 20 μg/kg and 200 μg/kg. Thymalfasin has been found to be safe for humans when administered in doses as high as 16 mg/dose/day, and in rats as high as 6 mg/kg/day.

The thymalfasin can be administered by any of a variety of means well-known in the art, for example by injection or infusion intravenously, interperitoneally, intramuscularly or directly into the peri-tumoral area. In preferred embodiments, the thymalfasin peptide is present in a pharmaceutically acceptable liquid carrier, such as water for injection, saline in physiologic concentrations, or similar. The plasma half-life of subcutaneously injected thymalfasin is only about 2 hours. Rost, et al., 1998. However, conjugation of a polymer to a thymalfasin peptide substantially increases the plasma half-life of the peptide. Rasi, et al. (unpublished observations). When applied in the context of a conjugated thymalfasin, the above dosages reflect only the thymalfasin peptide present in the composition, and not the weight of the polymer conjugated thereto.

The isolation, characterization and use of thymalfasin peptides is described, for example, in U.S. Pat. No. 4,079,127, U.S. Pat. No. 4,353,821, U.S. Pat. No. 4,148,788 and U.S. Pat. No. 4,116,951. The present invention is applicable to thymalfasin peptides including naturally occurring thymalfasin as well as synthetic thymalfasin and recombinant thymalfasin having the amino acid sequence of naturally occurring thymalfasin, amino acid sequences substantially similar thereto, or an abbreviated sequence form thereof, and their biologically active analogs having substituted, deleted, elongated, replaced, or otherwise modified sequences which possess bioactivity substantially similar to that of thymalfasin, e.g., a thymalfasin peptide having sufficient amino acid homology with thymalfasin such that it functions in substantially the same way with substantially the same activity as thymalfasin.

### EXAMPLE 1

### In Vitro

WiDr (human), IA-XsSBR (rat), and DHD-K12 (rat) colorectal cancer cell lines were treated with thymalfasin from 6 to 48 hours at 5-100 μg/ml. TLP antigen expression was determined by flow cytometry (FC) and antigen localization by confocal laser scanning microscopy (CLSM), using TLP antiserum raised against a 9 amino acid peptide epitope of TLP (CSH-275).

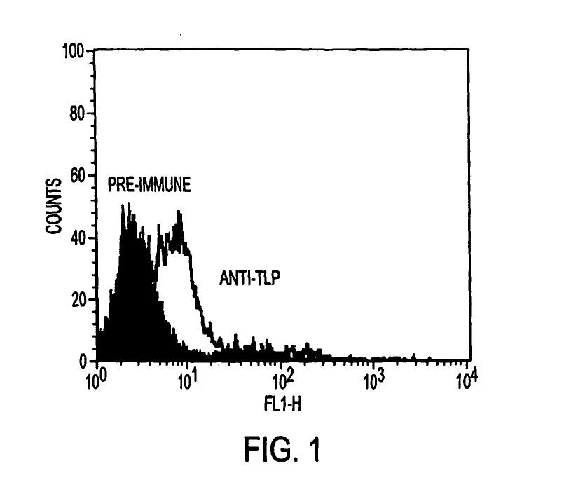

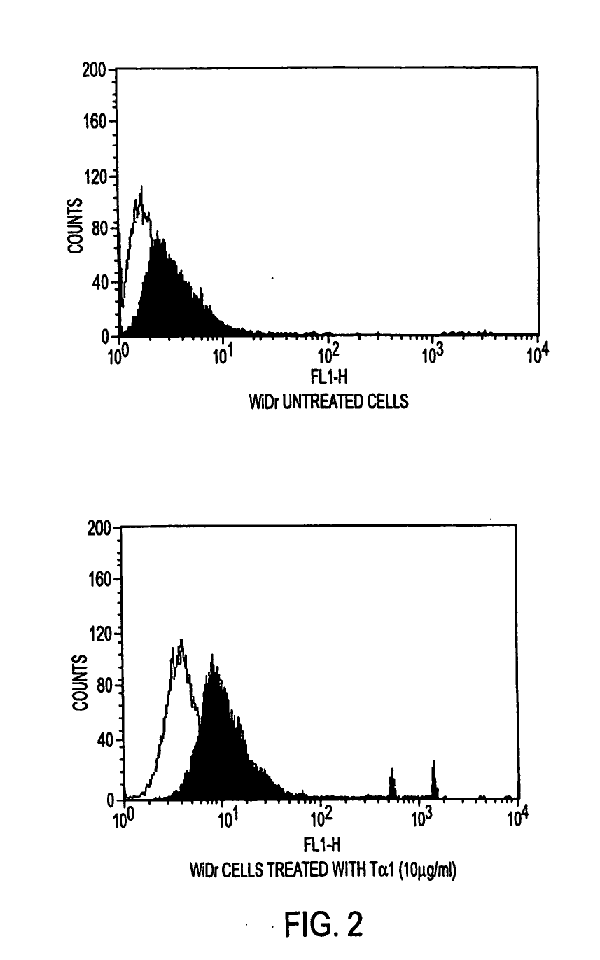

TLP is naturally expressed in all 3 colorectal cell lines, both in the cytoplasm, ranging from 30-55% of cells (by CLSM and by FC after permeabilization) and on the cell membrane, ranging from 10-20% of cells (by FC). FIG. 1. Thymalfasin is able to enhance the expression of this antigen in all cell lines tested. FIG. 2. The level of enhancement, and localization at the membrane versus the cytoplasm, varies with dosage and timing of treatment, and can reach expression levels of 90% of cells.

### EXAMPLE 2

### In Vivo

BD-IX rats were injected i.p. with syngeneic DHD-K12 cells and treated i.p. or s.c. with thymalfasin. TLP expression and localization were determined as above on tumor cells obtained both from ascites or tumor mass.

Tumor cells from animals treated with thymalfasin demonstrate an enhancement of expression of TLP similar to that seen in vitro. FIG. 3.

### REFERENCES

Andreone P., Cursaro C., Gramenzi A., et al. “A randomized controlled trial of thymosin alpha 1 versus interferon alpha treatment in patients with hepatitis B e antigen antibody—and hepatitis B virus DNA—positive chronic hepatitis B,” Hepatology 24(4): 774-777 (1996).
Baxevanis, C. N., et al., “Enhancement of human T lymphocyte function by prothymosin alpha: increased production of interleukin-2 and expression of interleukin-2 receptors in normal human peripheral blood T lymphocytes,” Immunopharmacol. Immunotoxicol. 12(4):595-617 (1990).
Bepler, G., “Thymosin alpha-1 as adjunct for conventional therapy of malignant tumors: a review,” Cancer Invest. 12:491-6 (1994).
Garaci, E., et al. “A randomized controlled study for the evaluation of the activity of a triple combination of zidovudine, thymosin alpha 1, and interferon alpha in HIV-infected individuals with CD4 counts between 200 and 500 cells/mm³,” Antiviral Ther. 3: 103-111(1998)
Garaci, E., F. Pica, G. Rasi, and C. Favalli, “Thymosin alpha 1 in the treatment of cancer: from basic research to clinical application,” Int. J. Immunopharm. 22: 1067-1076 (2000).
Giuliani, C., G. Napolitano, A. Mastino, S. Da Vincenzo, C. D'Agostini, S. Grelli, I. Bucci, D. S. Singer, L. D. Kohn, F. Monaco, E. Garaci & C. Favalli, “Thymosin-α1 regulates MHC class I expression in FRTL-5 cells at transcriptional level,” Eur. J. Immunol. 30:778-86 (2000).
Gravenstein S., Ershler W. B., Drumaskin S., Schwab R., Weksler M. E., “Anti-influenza antibody response: augmentation in elderly “non-responders” by thymosin alpha 1,” Gerontologist 26: 150A (1986).
Mutchnick, M. G. et al., “Thymosin alpha 1 treatment of chronic hepatitis B: results of a phase III multicentre, randomized, double-blind and placebo-controlled study,” J. Viral Hep. 6: 397-403 (1999).
Rost, K. L., W. Wierich, F. Masayuki, C. W. Tuthill and W. M. Herrmann, “Pharmacokinetics of thymosin α-1 after subcutaneous injection of three different formulations in healthy volunteers,” Eur. J. Clin. Pharmacol. 37:51-57 (1998).
Serrate S., Schulof R., Leondaridis L., Goldstein A. L., Sztein M. B., “Modulation of human natural killer cell cytotoxic activity, lymphokine production, and interleukin 2 receptor expression by thymic hormones”, J. Immunol. 139: 2338-2343 (1987).
Sherman, K. E. and Sherman, S. N. “Interferon plus thymosin alpha 1 treatment of chronic hepatitis C infection: a meta-analysis,” in Therapies for Viral Hepatitis. Schinazi, R F, Sommadossi, J-P, and Thomas, H C, editors. International Medical Press, 379-383 (1998).
Sztein M. and Serrate S, “Characterization of the immunoregulatory properties of thymosin alpha 1 on interleukin-2 production and interleukin-2 receptor expression in normal human lymphocytes”, Int. J. Immunopharmacol. 11:789-800 (1989).

1-10. (canceled)

11. A method enhancing expression of tumor antigen comprising treating tumor cells with an amount of thymalfasin sufficient to increase expression of tumor antigen relative to that of untreated tumor cells.

12. The method of claim 11, wherein the administration is to a patient in vivo.

13. The method of claim 12, wherein the thymalfasin is administered at a dose between 2 μg/kg body weight and 6 mg/kg body weight.

14. The method of claim 13, wherein the thymalfasin is administered at a dose between 20 μg/kg body weight and 200 μg/kg body weight.

15. The method of claim 11 wherein said tumor antigen is TLP.

16. A method for enhancing the sensitivity of a immunodiagnostic or immunotherapeutic method, comprising pre-treating target tumor cells by administering to the cells an amount of thymalfasin sufficient to increase the expression of tumor antigen relative to that of untreated tumor cells, followed by application of the immunodiagnostic or immunotherapeutic method.

17. The method of claim 16, wherein the administration is to a patient in vivo.

18. The method of claim 17, wherein the thymalfasin is administered at a dose between 2 μg/kg body weight and 6 mg/kg body weight.

19. The method of claim 18, wherein the thymalfasin is administered at a dose between 20 μg/kg body weight and 200 μg/kg body weight.

20. The method of claim 11 wherein the administration is to cells in vitro.

21. The method of claim 16 wherein the administration is to cells in vitro.

22. The method of claim 16 wherein said tumor antigen is TLP.
